(19) United States
(12) Patent Application Publication
(10) Pub. No.: US 2021/0052687 A1
(43) Pub. Date: Feb. 25, 2021

(54) PLANT EXTRACT-BASED BIOLOGICALLY ACTIVE SUPPLEMENT HAVING ANTIBACTERIAL, ANTIVIRAL AND ANTIFUNGAL ACTION

(71) Applicant: "BIO 8", Limited Liability Company, Moscow (RU)

(72) Inventors: Anton Lvovich Prozumentov, Moscow (RU); Georgiy Viktorovich Sarychev, Moscow (RU); Vera Konstantinovna Vedmina, Moscow (RU); Ivan Sergeevich Martynenko, Barnaul, Altai region (RU)

(21) Appl. No.: 16/331,508

(22) PCT Filed: Jul. 24, 2018
(86) PCT No.: PCT/RU2018/000490

§ 371 Date: Mar. 7, 2019

(51) Int. Cl. A61K 36/539; A61K 9/00; A61K 36/29; A61K 36/28; A61K 36/062; A61K 36/185; A61K 9/28; A61P 31/04; A61P 31/10

(52) U.S. Cl. CPC A61K 36/539; A61K 9/0053; A61K 36/29; A61K 36/28; A61K 36/062; A61P 31/10; A61K 9/2813; A61K 9/2866; A61K 9/2853; A61P 31/04; A61K 36/185

(57) ABSTRACT

The present invention relates to a biologically active supplement formulated in an oral dosage form comprising nutraceutically effective amounts of an extract of Usnea barbata lichen, three-lobe beggarticks herb and common barberry berries and having an over-cumulative antibacterial, including bactericidal, action against the wide range of pathogens and conditional human pathogens, as well as over-cumulative antifungal action, including fungistatic and fungicidal actions. In addition, the supplement can comprise black elder berry extract, Baikal skullcap root extract, and fermented black garlic powder.

The biologically active supplement of the invention can be used for enhancement of the general non-specific resistance of the body and for prevention or adjuvant therapy of the wide range of bacterial and fungal infections.

### FIELD OF THE INVENTION

The invention relates to the field of chemical, pharmaceutical and food industries, in particular to a biologically active supplement having a wide spectrum of antibacterial, antiviral and antifungal, including fungicidal, action encompassing many bacterial, viral and fungal pathogens important for humans, the supplement comprising a combination of plant extracts.

### BACKGROUND

One of the important directions in the development of new agents designed to affect bacterial, viral and fungal pathogens and conditional pathogens of importance in medicine is creating the new biologically active supplements, which currently represent an important alternative to traditionally used drugs.

In creating the new formulations of biologically active supplements having antibacterial, antiviral and antifungal action, it is advantageous to use the medicinal plant potential and to identify new types of activity not previously studied in already known medicinal plants. One of these plants is three-lobe beggarticks (Bidens tripartita), an annual herbaceous plant of the sunflower family distributed in the most part of Eurasia, North Africa, and North America. Traditionally, three-lobe beggarticks is used in traditional medicine as a diaphoretic, diuretic and antiallergic agent, see in “Phytopharmacognostic dictionary (Russian: [symbol] - [symbol] [symbol] )” edited by K. F. Blinova and G. P. Yakovleva.-M., “Higher School,” 1990, p. 256)), however Tomczykowa M., Tomczyk M., Jakoniuk P., Tryniszewska E. “Antimicrobial and antifungal activities of the extracts and essential oils of Bidens tripartita”—“Folia Histochemica et Cytobiologica,” 2008, volume 46, No. 3, pp. 389-393 (hereinafter as Tomczykowa M., Tomczyk M., et al., 2008) reports that three-lobe beggarticks herb extracts also have antibacterial activity. In particular, Tomczykowa M., Tomczyk M., et al., 2008 shows that three-lobe beggarticks herb extracts are active against many Gram-positive microorganisms, e.g., against Staphylococcus aureus, and against certain Gram-negative microorganisms such as Escherichia coli including Escherichia coli strains producing β-lactamase, Klebsiella pneumoniae including Klebsiella pneumoniae strains producing extended-spectrum β-lactamases, and against Pseudomonas aeruginosa.

Further, Tomczykowa M., Tomczyk M. et al., 2008 shows the fungistatic action of essential oils of three-lobe beggarticks herb particularly against Candida albicans, Candida parapsilosis, Aspergillus fumigatus and Aspergillus terreus. However, Tomczykowa M., Tomczyk M. et al., 2008 reports that three-lobe beggarticks herb extract or any separate components thereof can have a fungicidal action against Candida albicans, Candida parapsilosis, Aspergillus fumigatus, Aspergillus terreus or other fungal pathogens or conditional pathogens in human.

Also, Tomczykowa M., Tomczyk M. et al., 2008 reveals no combined compositions comprising three-lobe beggarticks herb extracts wherein the three-lobe beggarticks herb action could be enhanced quantitatively or varied qualitatively with the expanding the spectrum of antibacterial or antifungal action when three-lobe beggarticks herb extract is combined with another antibacterial or antifungal active ingredient, e.g., with antibacterial or antifungal active plant extracts. Finally, Tomczykowa M., Tomczyk M. et al., 2008 doesn't disclose that extracts or essential oils of the three-lobe beggarticks herb have the bacteriostatic or bactericidal activity against Salmonella enteritidis, Acinetobacter baumannii, Haemophylus influenzae and Enterobacter cloacae.

Another important ingredient that could enhance the antibacterial and antifungal (fungistatic) properties of three-lobe beggarticks herb extract and expand the spectrum of antibacterial action is common barberry (Berberis vulgaris), more specifically common barberry berry extract.

Common barberry is a shrub in the barberry family (Berberidaceae), which grows in West Asia, Transcaucasia, and in Central, Eastern and Southern Europe. In Russia, common barberry grows in the European part, mainly in the forest-steppe zone.

Usage of common barberry for treating gastrointestinal tract, liver, gallbladder, kidneys, urinary system and respiratory system diseases, as well as for treating cardiovascular diseases is known in the art (Blumenthal M., Busse W., Goldberg A., Gruenwald J., Hall T. R. C., Rister R. “The Complete German Commission E Monographs: Therapeutic Guide to Herbal Medicines”—“American Botanical Council,” 1^{st} edition (Oct. 1, 1998), p. 80).

Usage of the extracts of common barberry and other plants in the barberry family (including Berberis aquifolium and Berberis aristata) for treating the chronic inflammatory processes is also known in the art (Ivanovska N., Philipov S. “Study on the anti-inflammatory action of Berberis vulgaris root extract, alkaloid fractions and pure alkaloids”—“International Journal of Immunopharmacology,” 1996, volume 18, pp. 553-561). Antioxidant activity is also shown to present in different members of the barberry family (Zovko Konči\ddot{c} M., Kremer D., Karlović K., Kosalec I. “Evaluation of antioxidant activities and phenolic content of Berberis vulgaris and Berberis croatica”—“Food and Chemical Toxicology,” 2010, volume 48, Nos. 8-9, pp. 2176-2180).

Ghareeb A., Abeer E. et al. “Biological assessment of Berberis vulgaris and its active constituent, berberine: Antibacterial, antifungal and anti-hepatitis C virus (HCV) effect”—“Journal of Medicinal Plant Research,” 2013, volume 7, No. 21, pp. 1529-1536 (hereinafter as Ghareeb A., Abeer E. et al., 2013) reported that barberry berry extract has antibacterial action against Escherichia coli and fungistatic action against Candida albicans. Ghareeb A., Abeer E. et al., 2013 as well as other prior art references, however, describes no fungicidal action of barberry berry extract.

Further, Ghareeb A., Abeer E. et al., 2013 doesn't contemplate a possibility of enhancing the antibacterial and/or antifungal properties of barberry berry extract by combining thereof with other antibacterial or antifungal active plant extracts.

Zahra Dashti, Nabi Shariatifar, and Abdolreza Mohammadi Nafchi. “Study on antibacterial and antioxidant activity of Berberis vulgaris aqueous extracts of Iran”—“International Journal of Pharma Sciences and Research,” volume 5, No. 10, October 2014, pp. 705-708 (hereafter as Zahra Dashti et al., 2014) reveals that common barberry berry extracts exhibit the antibacterial activity against certain Gram-positive bacteria including Staphylococcus aureus and Bacillus cereus, and against certain Gram-negative bacteria (e.g., against Escherichia coli).

However, Zahra Dashti et al., 2014 and Ghareeb A., Abeer E. et al., 2013 do not contemplate a possibility of potentiating the antibacterial or antifungal action of barberry berry extract or expanding the spectrum of antibacterial or antifungal action of barberry berry extract by combining thereof with other plant extracts. Also, Zahra Dashti et al., 2014 and Ghareeb A., Abeer E. et al., 2013 do not contemplate a possibility of creating biologically active supplements comprising barberry berry extract and having an enhancing action for the non-specific resistance of the body as well as a wide spectrum of the antibacterial and antifungal action. Finally, Zahra Dashti et al., 2014 and Ghareeb A., Abeer E. et al., 2013 do not suggest a possibility of providing a fungicidal effect of barberry berry extract in particular when combined with other plant extracts having an antifungal effect.

Another important ingredient, which potential can be used for enhancing the antibacterial and antifungal properties of common barberry berries and three-lobe beggarticks herb and for creating biologically active supplements having an antibacterial and antifungal action, is Usnea barbata lichen (Usnea barbata) containing usnic acid having antibacterial properties. Usage of usnic acid salt sodium usninate for treating infected wounds, trophic ulcers, and burns is also well-known in the art (see “Phytopharmacognostic dictionary” edited by K. F. Blinova and G. P. Yakovleva.-M., “Higher School,” 1990, p. 256).

Madamombe I. T., Afolayan A. J. “Evaluation of antimicrobial activity of extracts of South African Usnea barbata”—“Pharmaceutical Biology,” 2003, volume 41, No. 3, pp. 199-202 (hereinafter as Madamombe I. T., Afolayan A. J., 2003) reports that Usnea barbata extracts have an antibacterial action against Gram-positive microorganisms such as Bacillus subtilis, Enterococcus faecalis, Micrococcus viridans and Staphylococcus aureus, and against certain Gram-negative microorganisms (Escherichia coli and Pseudomonas aeruginosa). Also, Madamombe I. T., Afolayan A. J., 2003 showed the fungistatic action of Usnea barbata extract. Meanwhile, Madamombe I. T., Afolayan A. J., 2003 doesn't disclose that Usnea barbata extract has a fungicidal action; and doesn't show that the antibacterial and/or antifungal action of Usnea barbata extract can be enhanced when the extract is combined with other antibacterial or antifungal active ingredients, e.g., with antibacterial or antifungal active plant extracts.

Published patent application US 2007/0154581 A1 “Composition and method for enhancing or stimulating the immune system,” by: KUMAR KALYANI M (US), GRANTE JULLIAN IRVING (US) and MCCLINTOCK ROBERT (US), IPC: A61K 36/185, A61K 31/76, A61K 31/315, A61K 31/095, published on Jul. 5, 2007, discloses the closest prior art biologically active food supplement for increasing resistance of the human recipient body to a wide range of infectious pathogens including bacterial, viral and fungal pathogens, the supplement comprising elderberry extract and allicin. Allicin is known to be organic sulfoxide formed during the disintegration of garlic cells and determines, as shown in Cavallito C., Bailey J. H. “Allicin, the antibacterial principle of Allium sativum. Isolation, physical properties and antibacterial action”—“Journal of the American Chemical Society,” 1944, volume 66, pp. 1944-1952 and Yamada Y., Azuma K. “Evaluation of the in vitro antifungal activity of allicin”—“Antimicrobial Agents and Chemotherapy,” 1977, volume 11, No. 4, pp. 743-747, antibacterial, antiviral and antifungal (fungistatic) properties of garlic.

Preferably, the supplement according to US 2007/0154581 A1 is formulated in an oral dosage form such as a capsule or tablet containing excipients suitable for the manufacture of oral dosage forms (see paragraph [0122] in the specification of US 2007/0154581 A1). As an example of such excipients, US 2007/0154581 A1 teaches unmodified corn maltodextrin, cellulose, rice flour, magnesium stearate and silicon dioxide (see paragraphs [0119] and [0121] in the specification of US 2007/0154581 A1).

The closest prior art supplement has a tonic action for the human immune system, and according to inventors, should have a wide spectrum of antiviral, antibacterial and antifungal activity including antibacterial activity against Escherichia, Salmonella, Staphylococcus, Streptococcus, Klebsiella, Clostridium and other important bacterial pathogens, antiviral activity against human cytomegalovirus, influenza B virus, herpes simplex virus type I, herpes simplex virus II type, parainfluenza virus type III, vesicular stomatitis virus and antifungal (fungistatic) activity against Cryptococcus neoformans, Candida, Trichophyton, Epidermphyton and Microsporum. However, the supplement according to US 2007/0154581 A1 was acknowledged as effective only for the prevention of colds.

Since the possibility of using the supplement for prevention and/or adjuvant therapy of bacterial and/or fungal infections have not been shown, there is still a need to develop a plant extract-based nutritional supplement formulation, wherein using of the supplement would enhance the overall non-specific resistance of the recipient's body, and the supplement, having a wide spectrum of antibacterial and antifungal action, could be used for prevention or adjuvant therapy of bacterial, fungal and viral infections.

In addition, still of great relevance is to expand the range of plant extract-based biologically active supplements having antibacterial, including bacteriostatic and bactericidal, action against the wide range of microorganisms including Escherichia coli (in particular against strains of Escherichia coli, Staphylococcus aureus, Klebsiella pneumoniae, Salmonella enteritidis, Pseudomonas aeruginosa, Enterococcus faecalis, Acinetobacter baumannii, Streptococcus pneumoniae, Enterobacter cloacae, Streptococcus pyogenes, Haemophylus influenzae and Streptococcus agalactiae and antifungal (at least fungistatic) action against Candida albicans. Also, there is still need to provide an antiviral action of such supplement primarily against influenza A and B viruses and rhinoviruses. This problem is especially important due to the common usage of antibiotics promoting the development of antibiotic-resistant strains of pathogenic and opportunistic microorganisms. Among such antibiotic-resistant microorganisms that are of clinical importance, it should be noted strains and isolates of Escherichia coli producing β-lactamases, such as expanded spectrum β-lactamases (BLES-producing Escherichia coli), methicillin-resistant aurococcus (Staphylococcus aureus) (MRSA), ESBL-producing Klebsiella pneumoniae, blue pus bacillus (Pseudomonas aeruginosa) producing metal-β-lactamases, and ESBL-producing Enterobacter cloacae. Also, there is still need to expand the range of agents having a wide spectrum of antiviral effects, especially those that are active against influenza A and B viruses and against rhinoviruses.

Finally, as discussed, the prior art has not been able to provide a fungicidal effect using plant monoextracts of three-lobe beggarticks herb, common barberry berries, Usnea barbata, black elderberry berries, Baikal skullcap roots or combinations thereof, therefore there is still a need to create a plant extract-based biologically active supplement that would have a fungicidal action and could be used for prevention or complex therapy against candida infections.

### SUMMARY OF THE INVENTION

These problems have been solved within the present invention by creating the biologically active supplement comprising a combination of extracts of Usnea barbata lichen, three-lobe beggarticks herb, common barberry berries, black elderberry, Baikal skullcap and optionally black garlic. The technical result of the claimed invention consists firstly in expanding the range of biologically active supplements increasing the overall non-specific resistance of the body, having antibacterial, antiviral and antifungal action, and secondly in achieving an over-cumulative (synergistic) action, in particular antibacterial (bacteriostatic and bactericidal) and antifungal (fungistatic and fungicidal) effect against the wide range of pathogens and conditional pathogens of importance in medicine, and in expanding the spectrum of said biologically active supplement action.

The object and the result are achieved within the present invention by creating a biologically active supplement formulated in an oral dosage form of an antibacterial and antifungal action comprising nutraceutically effective amounts of common barberry berry extract, three-lobe beggarticks herb extract, Usnea barbata lichen extract, black elderberry berry extract, Baikal skullcap extract and optionally black garlic combined with nutraceutically acceptable excipients. The present biologically active supplement within the context of the present invention is shown to be active against the wide range of human pathogens and conditional pathogens including Escherichia coli, Staphylococcus aureus, Klebsiella pneumoniae, Salmonella enteritidis, Pseudomonas aeruginosa, Enterococcus faecalis, Acinetobacter baumannii, Streptococcus pneumoniae, Streptococcus pyogenes, Haemophylus influenzae, Streptococcus agalactiae, Enterobacter cloacae, Candida albicans, influenza A and B viruses and rhinoviruses, the supplement administration providing over-cumulative antibacterial (bacteriostatic and bactericidal) and over-cumulative antifungal (fungistatic and fungicidal) effects as well as antiviral effect preferably against influenza A and B viruses and against rhinoviruses. Within the present invention, the antiviral effect is achieved by including black elderberry berry extract and Baikal skullcap root extract into the composition of said biologically active supplement. This allows using the biologically active supplement of the invention for increasing the overall non-specific resistance of the recipient body and for prevention and adjuvant therapy of said infections.

### PREFERRED EMBODIMENTS OF THE INVENTION

In preferred (non-limiting) embodiments of the present invention, standardized extracts of common barberry berries, three-lobe beggarticks herb and Usnea barbata, black elderberry berries, Baikal skullcap roots and standardized fermented black garlic powder successfully passed internal qualitative control are used.

In addition, in preferred (non-limiting) embodiments of the present invention, the biologically active supplement of the invention can be formulated in an oral dosage form. More preferably, but not limited to these embodiments, the biologically active supplement of the invention is formulated in an oral solid dosage form. Most preferably, but not limited to the embodiments, the biologically active supplement of the invention is formulated in a tablet, or capsule, or powder, or granules containing, in addition to the extracts of beggars-ticks, barberry, Usnea, elderberry and skullcap, nutraceutically acceptable excipients including but not limited to colloidal silicon dioxide, talc and calcium stearate, or magnesium stearate as free flowing agents; and microcrystalline cellulose or sugar, e.g., but not limited to, sucrose, fructose or lactose, as an excipient (vehicle). Specifically, sugars are preferably used as a vehicle for powder and granular form. Also, in certain non-limiting embodiments, sugars can be used as excipients in the manufacture of tablets or capsules of the biologically active supplement. As microcrystalline cellulose, commercially available microcrystalline celluloses can be used, such as, e.g., Avicel®, Vivapur® or the like.

In the most preferred (non-limiting) embodiments of the present invention, the biologically active supplement of the invention is formulated as a film-coated tablet. The tablet coat is made of a commercially available tablet coating mixture. An exemplary (non-limiting) suitable coating could be a commercially available tablet coating mixture, such as commercially available mixture Opadry® consisting of hydroxypropyl methylcellulose, titanium dioxide, polyethylene glycol and talc, or similar mixtures. Those ordinary skilled in the art would understand that the exemplary suitable coating is non-limiting and that other commercially available tablet coating mixtures could be used to prepare the tablet coating.

In one of the most preferred (non-limiting) embodiments of the present invention, the tablet of the claimed biologically active supplement is film-coated and consists of a core comprising from 0.5% to 90.0% of Usnea barbata lichen extract, from 0.5% to 90.0% of common barberry berry extract, and from 0.5% to 90.0% of three-lobe beggarticks herb extract, from 0.5% to 90.0% of black elderberry berries extract, from 0.5% to 90.0% combined with nutraceutically acceptable excipients such as calcium stearate or magnesium stearate, talc, amorphous (colloidal) silicon dioxide and microcrystalline cellulose, and a coat comprising hydroxypropyl methylcellulose, titanium dioxide, polyethylene glycol and talc.

In one of the most preferred non-limiting embodiments, the tablet consists of a core containing an extract mixture comprising preferably 0.001 to 900.0 mg of Usnea barbata lichen extract, 0.001 to 900.0 mg of common barberry berry extract, 0.001 to 900.0 mg of three-lobe beggarticks herb extract, 0.001 to 900.0 mg of black elderberry extract, 0.001 to 900.0 mg of Baikal skullcap extract and optionally 0.001 to 900.0 mg of black (fermented) garlic powder, and the required amount of auxiliary ingredients, such as but not limited to microcrystalline cellulose, calcium stearate or magnesium stearate, talc and colloidal silicon dioxide, and a film coat over the core made of hydroxypropyl methylcellulose, titanium dioxide, polyethylene glycol and talc.

In the alternative most preferred embodiments of the present invention, the biologically active supplement of the invention is formulated in a hard gelatin capsule, e.g., in a hard gelatin capsule comprising a mixture, or in other dosage forms suitable for oral use, such as dragee, pills, powders, granules, etc. If necessary, the biologically active supplement of the invention can be formulated in oral dosage forms characterized by a modified release of the active substances.

In the most preferred embodiments of the invention, excipients of pharmacopeia grade are used to prepare the biologically active supplement of the invention. (Hereinafter, excipients of pharmacopeia grade are excipients that meet requirements of the European Pharmacopoeia or the State Pharmacopoeia of the Russian Federation XIII edition.)

In alternative embodiments of the present invention, other excipients of pharmacopeia grade commonly used for the manufacture of solid dosage forms can be used instead of microcrystalline cellulose, colloidal silicon dioxide, and calcium stearate or magnesium stearate. A non-limiting (illustrative) example of such excipients can be excipients indicated as useful for the manufacture of solid dosage forms in Raymond C. Rowe, Paul J. Sheskey, Marian E. Quinn. “Handbook of Pharmaceutical Excipients”—“Pharmaceutical Press,” 2009 or in similar guidelines.

The present invention is illustrated by the following examples, which however are intended only to illustrate the invention and not to limit the scope of the claims.

### EXAMPLES

### Example 1: Inventive Biologically Active Supplement Preparing as Capsules

The raw material for the production of inventive biologically active supplement capsules—three-lobe beggarticks herb extract in the amount of 900.0 g, Usnea barbata lichen extract in the amount of 1.0 g and common barberry berry extract in the amount of 1.0 g, black elder berry extract in the amount of 1.0 g, Baical skullcap root extract in the amount of 1.0 g—is loaded into a reactor. Excipients including 71.0 g of microcrystalline cellulose (Avicel® or equivalent), 5.0 g of colloidal silicon dioxide (Aerosil® or equivalent), 10.0 g of calcium stearate and 10.0 g of talc are added to the reactor and stirred to form a capsule filling mixture. The resulting mixture is portioned into 1000 equal portions for subsequent capsule filling using an encapsulation machine, loaded into hard gelatin capsules (1000 pieces). The resulting capsules are sealed, dedusted and packaged in jars or blisters.

### Example 2: Inventive Biologically Active Supplement Preparing as Capsules

The raw material for the production of inventive biologically active supplement capsules—three-lobe beggarticks herb extract in the amount of 1.0 g, Usnea barbata lichen extract in the amount of 900.0 g and common barberry berry extract in the amount of 1.0 g, black elder berry extract in the amount of 1.0 g, Baical skullcap root extract in the amount of 1.0 g—is loaded into a reactor. Excipients including 71.0 g of microcrystalline cellulose (Avicel® or equivalent), 5.0 g of colloidal silicon dioxide (Aerosil® or equivalent), 10.0 g of calcium stearate and 10.0 g of talc are added to the reactor and stirred to form a capsule filling mixture. The resulting mixture is portioned into 1000 equal portions for subsequent capsule filling using an encapsulation machine, loaded into hard gelatin capsules (1000 pieces). The resulting capsules are sealed, dedusted and packaged in jars or blisters.

### Example 3: Inventive Biologically Active Supplement Preparing as Capsules

The raw material for the production of inventive biologically active supplement capsules—three-lobe beggarticks herb extract in the amount of 1.0 g, Usnea barbata lichen extract in the amount of 1.0 g and common barberry berry extract in the amount of 900.0 g, black elder berry extract in the amount of 1.0 g, Baical skullcap root extract in the amount of 1.0 g—is loaded into a reactor. Excipients including 71.0 g of microcrystalline cellulose (Avicel® or equivalent), 5.0 g of colloidal silicon dioxide (Aerosil® or equivalent), 10.0 g of calcium stearate and 10.0 g of talc are added to the reactor and stirred to form a capsule filling mixture. The resulting mixture is portioned into 1000 equal portions for subsequent capsule filling using an encapsulation machine, loaded into hard gelatin capsules (1000 pieces). The resulting capsules are sealed, dedusted and packaged in jars or blisters.

### Example 4: Inventive Biologically Active Supplement Preparing as Capsules

The raw material for the production of inventive biologically active supplement capsules—three-lobe beggarticks herb extract in the amount of 1.0 g, Usnea barbata lichen extract in the amount of 1.0 g and common barberry berry extract in the amount of 1.0 g, black elder berry extract in the amount of 900.0 g, Baical skullcap root extract in the amount of 1.0 g—is loaded into a reactor. Excipients including 71.0 g of microcrystalline cellulose (Avicel® or equivalent), 5.0 g of colloidal silicon dioxide (Aerosil® or equivalent), 10.0 g of calcium stearate and 10.0 g of talc are added to the reactor and stirred to form a capsule filling mixture. The resulting mixture is portioned into 1000 equal portions for subsequent capsule filling using an encapsulation machine, loaded into hard gelatin capsules (1000 pieces). The resulting capsules are sealed, dedusted and packaged in jars or blisters.

### Example 5: Inventive Biologically Active Supplement Preparing as Capsules

The raw material for the production of inventive biologically active supplement capsules—three-lobe beggarticks herb extract in the amount of 1.0 g, Usnea barbata lichen extract in the amount of 1.0 g and common barberry berry extract in the amount of 1.0 g, black elder berry extract in the amount of 1.0 g, Baical skullcap root extract in the amount of 900.0 g—is loaded into a reactor. Excipients including 71.0 g of microcrystalline cellulose (Avicel® or equivalent), 5.0 g of colloidal silicon dioxide (Aerosil® or equivalent), 10.0 g of calcium stearate and 10.0 g of talc are added to the reactor and stirred to form a capsule filling mixture. The resulting mixture is portioned into 1000 equal portions for subsequent capsule filling using an encapsulation machine, loaded into hard gelatin capsules (1000 pieces). The resulting capsules are sealed, dedusted and packaged in jars or blisters.

### Example 6: Inventive Biologically Active Supplement Preparing as Film-Coated Tablets

To the raw material for the inventive biologically active supplement preparation comprising three-lobe beggarticks herb extract in the amount of 900.0 g, Usnea barbata lichen extract in the amount of 1.0 g, common barberry berry extract in the amount of 1.0 g, black elder berry extract in the amount of 1.0 g and Baical skullcap root extract (or a Baikal skullcap root powder) in the amount of 1.0 g placed into a reactor, excipients, such as microcrystalline cellulose in the amount of 184 g, calcium stearate in the amount of 5.0 g, talc in the amount of 5.0 g, and amorphous (colloidal) silicon dioxide in the amount of 2.5 g, are added, mixed, formed as a tabletting mass, portioned into 1000 equal portions, and transferred to a tablet press to perform tabletting.

The produced tablets (1000 pcs.) are dedusted, film coated with a commercially available tablet coating composition Opadry® comprising hydroxypropyl methylcellulose, titanium dioxide, polyethylene glycol and talc. If necessary, the tablets are stamped with a logo or other marks. Film-coated tablets are packed in blisters or in vials.

### Example 7: Inventive Biologically Active Supplement Preparing as Film-Coated Tablets

Inventive biologically active supplement tablets are prepared in the same manner as in Examples Example 6 using, as the raw material for the inventive biologically active supplement preparation, three-lobe beggarticks herb extract in the amount of 1.0 g, Usnea barbata lichen extract in the amount of 900.0 g, common barberry berry extract in the amount of 1.0 g, black elder berry extract in the amount of 1.0 g, and Baical skullcap root extract, or a Baikal skullcap root powder, in the amount of 1.0 g. Fermented black garlic powder in the amount of 1.0 g is added thereto, mixed, formed as a tableting mass, portioned into 1000 equal portions, and transferred to a tablet press to perform tableting.

The produced tablets (1000 pcs.) are dedusted, film coated with a commercially available tablet coating composition Opadry® comprising hydroxypropyl methylcellulose, titanium dioxide, polyethylene glycol and talc. If necessary, the tablets are stamped with a logo or other marks. Film-coated tablets are packed in blisters or in vials.

### Example 8: Inventive Biologically Active Supplement Preparing as Film-Coated Tablets

Inventive biologically active supplement tablets are prepared in the same manner as in Examples 6 and 7 using, as the raw material for the inventive biologically active supplement preparation, three-lobe beggarticks herb extract in the amount of 1.0 g, Usnea barbata lichen extract in the amount of 1.0 g, common barberry berry extract in the amount of 900.0 g, black elder berry extract in the amount of 1.0 g, and Baical skullcap root extract, or a Baikal skullcap root powder, in the amount of 1.0 g. Tablets are produced, marked and packed in blisters or vials in the same way as described in Examples 6 and 7.

### Example 9: Inventive Biologically Active Supplement Preparing as Film-Coated Tablets

Inventive biologically active supplement tablets are prepared in the same manner as in Examples 6, 7 and 8 using, as the raw material for the inventive biologically active supplement preparation, three-lobe beggarticks herb extract in the amount of 1.0 g, Usnea barbata lichen extract in the amount of 1.0 g, common barberry berry extract in the amount of 1.0 g, black elder berry extract in the amount of 900.0 g, and Baical skullcap root extract, or a Baikal skullcap root powder, in the amount of 1.0 g. Tablets are produced, marked and packed in blisters or vials in the same way as described in Examples 6, 7 and 8.

### Example 10: Inventive Biologically Active Supplement Preparing as Film-Coated Tablets

Inventive biologically active supplement tablets are prepared in the same manner as in Examples 6, 7, 8 and 9 using, as the raw material for the inventive biologically active supplement preparation, three-lobe beggarticks herb extract in the amount of 1.0 g, Usnea barbata lichen extract in the amount of 1.0 g, common barberry berry extract in the amount of 1.0 g, black elder berry extract in the amount of 1.0 g, and Baical skullcap root extract, or a Baikal skullcap root powder, in the amount of 900.0 g. Tablets are produced, marked and packed in blisters or vials in the same way as described in Examples 6, 7, 8 and 9.

### Example 11: Study of Antibacterial and Antifungal Activity in Inventive Biologically Active Supplement

Study of antibacterial and antifungal activity in the inventive biologically active supplement was performed according to GOST R ISO 20776-1-2010 “Clinical laboratory studies and diagnostic test systems in vitro. Study of the sensitivity of infectious agents and evaluation of functional specifications for the study of sensitivity to antimicrobial means. Part 1. Reference method laboratory studies of the activity of antimicrobial agents against rapidly growing aerobic bacteria causing infectious diseases.” and according to the Guidelines MUK 4.2.1890-04 “Susceptibility Testing of Microorganisms to Antibacterial Agents” (approved and effective Mar. 4, 2004).

During the study, the antimicrobial activity of the inventive biologically active supplement comprising a combination of Usnea barbata lichen, common barberry berry, and three-lobe beggarticks herb extracts was evaluated for antibiotic-sensitive and antibiotic-resistant microorganism strains of clinical significance. During the study, the minimum inhibitory (MIC) and minimum bactericidal (MBC) concentration as well as minimum inhibitory (MI_{F}C) and minimum fungicidal (MFC) concentrations were determined for the extracts of Usnea barbata lichen, common barberry berries and three-lobe beggarticks herb for Candida albicans.

The antibacterial and antifungal activity study of the inventive composition and the identification of MIC, MBC, MI_{F}C, and MFC were performed at the Department of Microbiology in Smolensk State Medical University at the Ministry of Healthcare of the Russian Federation. For the study, the following strains from the specialized collection and clinical isolates from the collection of the Scientific Research Institute of Antimicrobial Chemotherapy at the Smolensk State Medical Academy (hereinafter as SRIAC SSMA) were used: Salmonella enteritidis, strain 14028, Klebsiella pneumoniae, expanded-spectrum β-lactamase (ESBL) producing strain ATCC 700603, Acinetobacter baumannii, strain ATCC 19606, Streptococcus pneumoniae, strain ATCC 49619, Streptococcus pyogenes, strain ATCC19615, Streptococcus agalactiae, strain ATCC 12386, Enterococcus faecalis, strain ATCC 29212, Escherichia coli, strain ATCC 25922, Escherichia coli, ESBL producing strain ATCC 35218, Pseudomonas aeruginosa, strain ATCC 27853, Pseudomonas aeruginosa, metallo-beta-lactamase producing strain (MBL-producing Pseudomonas aeruginosa), Staphylococcus aureus, strain ATCC 25923, Staphylococcus aureus methicillin-resistant (MRSA), Haemophylus influenza, strain ATCC 49766, Enterobacter cloacae—ESBL producing clinical isolate, and Candida albicans, strain ATCC 10231.

Microbial enumeration test of a test sample was performed following the methods in Russia State Pharmacopoeia (XIII edition, volume 1, GPA.1.2.4.0002.15 “Microbiological purity”). Susceptibility testing of microorganisms to the test composition of the inventive biologically active supplement was performed following the broth serial dilution microtechnique in sterile polystyrene flat-bottomed 96-well plates (“Sarstedt,” Germany).

For the present study, stock solutions of the extractions from the composition of the invention were used followed by double dilution thereof:

0.2%-0.1%-0.05%-0.025%-0.0125%

2%-1%-0.5%-0.25%-0.125%

20%-10%-5%-2.5%-1.25%

The pre-extractions from the composition of the invention were filter sterilized. To control sterility, the filtered solutions were added to thioglycolate medium in a ratio of 1:10, followed by incubation at 35° C. for 48 hours, and followed by seeding on FMH agar to validate the absence of microbial growth.

Inoculum with the final composition of microbial cells: 5×10⁵ CFU/ml.

Nutrient media: Müller-Hinton broth, Müller-Hinton agar (M-H broth will be used for the microplating method in broth, M-H agar will be used for cultivation of the daily indicative bacterial culture and MBC detection (MFC for Candida albicans) in the extracts of Usnea barbata, three-lobe beggarticks, and common barberry.

Controls: the nutrient broth without microbial culture (negative control); the nutrient broth with microbial culture without plant preparation (positive control).

The determination of minimal inhibitory (MIC; MI_{F}C for Candida albicans) and minimal bactericidal concentrations (MBC; minimum fungicidal concentration, MFC, for Candida albicans) in extractions from the composition of the invention was performed by microdilution method in the broth in sterile polystyrene flat-bottomed 96-well plates (“Sarstedt,” Germany) Two-fold serial dilutions in the Müller-Hinton broth (BD, USA) were prepared from the stock solutions of plant extracts of Usnea barbata, three-lobe beggarticks herb and common barberry berries (0.2%, 2%, 20%) from 20% solution to 0.0125%. Bacterial suspensions having an optical density of 0.5 McFarland (1×10⁸ CFU/ml) were prepared from daily cultures of test microorganisms grown on M-H agar in the sterile isotonic solution. The adjusted inoculum was diluted in the broth to provide the final cell concentration of 5×10⁵ CFU/ml (between 2×10⁵ CFU/ml and 8×10⁵ CFU/ml). Transferring 0.1 ml of the standardized organism suspension to a tube containing 9.9 ml (at the dilution ratio of 1:100) of the broth yields a suspension having the cell concentration of 1×10⁶ CFU/ml while adding 5 μl of which to 100 μl of the antibacterial agent solution produced the final inoculum composition of 5×10⁵ CFU/ml. The plates were incubated in an incubator for 18 hours at 35° C. MIC was registered based on the absence of microorganism visible growth by comparing the experimental and control wells. For MBC determination, 10 μl of each well was plated onto a sector of a solid nutrient medium (M-H agar). Following the incubation for 24 hours at 35° C., the microorganism growth on the nutrient medium was evaluated.

The minimum concentration preventing microbial growth is indicated as the minimum inhibitory concentration (MIC; MI_{F}C for Candida albicans); the minimum concentration killing 99.9% of microorganisms (from the baseline) is indicated as the minimum bactericidal concentration (MBC; MFC for Candida albicans).

Dilution concentrations in extracts of Usnea barbata lichen, common barberry berries, and three-lobe beggarticks herb, and all subsequent concentrations and ratios thereof were adjusted, when necessary, during the study as new data become available. The study results of the antibacterial and antifungal activity in monoextracts of three-lobe beggarticks herb, common barberry berries, and Usnea barbata lichen are provided in Tables 1-3 below. The study results of the antibacterial and antifungal activity of the inventive composition are provided in Table 4.

TABLE 1
Antibacterial and antifungal activity in monoextract of three-lobe
beggarticks herb (Bidens tripartita).

| | MIC | MBC |
|---|---|---|
| | (MI_{F}C*), | (MFC*), |
| Pathogen | mg/ml | mg/ml |
| Escherichia coli ATCC 25922 | 2.0 | 4.0 |
| Escherichia coli (ESBL) ATCC 35218 | 4.0 | 10.0 |
| Klebsiella pneumonia (ESBL) 700603 | 8.0 | 10.0 |
| Enterobacter cloacae (ESBL) clinical isolate | 4.0 | 10.0 |
| Salmonella typhimurium 14028 | 1.0 | 2.0 |
| Pseudomonas aeruginosa ATCC 27853 | 2.0 | 4.0 |
| Pseudomonas aeruginosa (MBL) | 5.0 | 5.0 |
| Acinetobacter baumannii ATCC 19606 | 2.0 | 4.0 |
| Staphylococcus aureus ATCC 25923 | 0.2 | 1.0 |
| Staphylococcus aureus (MRSA) | 0.8 | 1.0 |
| Streptococcus agalactiae ATCC 12386 | 0.1 | 0.1 |
| Streptococcus pyogenes ATCC 19615 | 0.05 | 0.1 |
| Streptococcus pneumonia ATCC 49619 | 0.05 | 0.1 |
| Enterococcus faecalis ATCC 29212 | 1.0 | 2.0 |
| Haemophilus influenzae ATCC 49766 | 0.1 | 0.1 |
| Candida albicans ATCC 10231 | 0.01 | No |

*For Candida albicans (strain ATCC 10231)

TABLE 2
Antibacterial and antifungal activity in monoextract of common barberry
berries (Berberis vulgaris).

| | MIC | MBC |
|---|---|---|
| | (MI_{F}C*), | (MFC*), |
| Pathogen | mg/ml | mg/ml |
| Escherichia coli ATCC 25922 | 0.7 | 2.6 |
| Escherichia coli (ESBL) ATCC 35218 | 2.6 | 7.0 |
| Klebsiella pneumonia (ESBL) 700603 | 2.6 | 7.0 |
| Enterobacter cloacae (ESBL) clinical isolate | 2.6 | 7.0 |
| Salmonella typhimurium 14028 | 0.2 | 0.7 |
| Pseudomonas aeruginosa ATCC 27853 | 0.7 | 1.4 |
| Pseudomonas aeruginosa (MBL) | 2.6 | 2.6 |
| Acinetobacter baumannii ATCC 19606 | 1.4 | 1.4 |
| Staphylococcus aureus ATCC 25923 | 0.1 | 0.1 |
| Staphylococcus aureus (MRSA) | 0.2 | 1.4 |
| Streptococcus agalactiae ATCC 12386 | 0.01 | 0.01 |
| Streptococcus pyogenes ATCC 19615 | 0.1 | 0.1 |
| Streptococcus pneumonia ATCC 49619 | 0.1 | 0.1 |
| Enterococcus faecalis ATCC 29212 | 1.4 | 1.4 |
| Haemophilus influenzae ATCC 49766 | 0.7 | 0.7 |
| Candida albicans ATCC 10231 | 0.1 | No |

*For Candida albicans (strain ATCC 10231)

TABLE 3
Antibacterial and antifungal activity in monoextract of usnea barbata
lichen (Usnea barbata).

| | MIC | MBC |
|---|---|---|
| | (MI_{F}C*), | (MFC*), |
| Pathogen | mg/ml | mg/ml |
| Escherichia coli ATCC 25922 | 5 | 10 |
| Escherichia coli (ESBL) ATCC 35218 | 10 | 16 |
| Klebsiella pneumonia (ESBL) 700603 | 10 | 16 |
| Enterobacter cloacae (ESBL) clinical isolate | 10 | 16 |
| Salmonella typhimurium 14028 | 2.5 | 5 |
| Pseudomonas aeruginosa ATCC 27853 | 5 | 10 |
| Pseudomonas aeruginosa (MBL) | 10 | 16 |
| Acinetobacter baumannii ATCC 19606 | 10 | 10 |
| Staphylococcus aureus ATCC 25923 | 1.0 | 2.5 |
| Staphylococcus aureus (MRSA) | 5.0 | 5.0 |
| Streptococcus agalactiae ATCC 12386 | 0.5 | 1.0 |
| Streptococcus pyogenes ATCC 19615 | 0.5 | 1.0 |
| Streptococcus pneumonia ATCC 49619 | 1.0 | 1.0 |
| Enterococcus faecalis ATCC 29212 | 2.5 | 5.0 |
| Haemophilus influenzae ATCC 49766 | 5 | 10 |
| Candida albicans ATCC 10231 | 0.25 | No |

*For Candida albicans (strain ATCC 10231)

TABLE 4
Antibacterial and antifungal activity in the inventive composition.

| | MIC | MBC |
|---|---|---|
| | (MI_{F}C*), | (MFC*), |
| Pathogen | mg/ml | mg/ml |
| Escherichia coli ATCC 25922 | 0.1 | 0.05 |
| Escherichia coli (ESBL) ATCC 35218 | 0.25 | 0.1 |
| Klebsiella pneumonia (ESBL) 700603 | 0.25 | 0.1 |
| Enterobacter cloacae (ESBL) clinical isolate | 0.25 | 0.1 |
| Salmonella typhimurium 14028 | 0.05 | 0.025 |
| Pseudomonas aeruginosa ATCC 27853 | 0.1 | 0.05 |
| Pseudomonas aeruginosa (MBL) | 0.25 | 0.1 |
| Acinetobacter baumannii ATCC 19606 | 0.25 | 0.1 |
| Staphylococcus aureus ATCC 25923 | 0.1 | 0.05 |
| Staphylococcus aureus (MRSA) | 0.1 | 0.05 |
| Streptococcus agalactiae ATCC 12386 | 0.05 | 0.025 |
| Streptococcus pyogenes ATCC 19615 | 0.05 | 0.025 |
| Streptococcus pneumonia ATCC 49619 | 0.1 | 0.025 |
| Enterococcus faecalis ATCC 29212 | 0.05 | 0.025 |
| Haemophilus influenzae ATCC 49766 | 0.05 | 0.025 |
| Candida albicans ATCC 10231 | 0.25 | 0.1 |

*For Candida albicans (strain ATCC 10231)

The data provided in Tables 1 to 3 show that for Candida albicans (strain ATCC 10231) MI_{F}C for monoextracts of Usnea barbata lichen, three-lobe beggarticks herb, and common barberry berries was 0.25, 0.01 and 0.1 mg/ml, respectively. The fungicidal effect against Candida albicans (strain ATCC 10231) was not found in monoextracts of Usnea barbata, three-lobe beggarticks herb, and common barberry berries.

In contrast, the inventive biologically active supplement comprising a combination of extracts of Usnea barbata, three-lobe beggarticks herb and common barberry berries, from the data obtained within the present study presented in Table 4, surprisingly exhibited fungicidal activity against Candida albicans, with minimal fungicidal concentration (MFC) of 0.1 mg/ml, although such fungicidal activity could not be predicted from the prior art.

In addition, from the data obtained within the present study and presented in Table 4, the inventive biologically active supplement exhibits the over-cumulative antibacterial, including bactericidal, action against the wide range of Gram-positive and Gram-negative microorganisms, such as against Salmonella Salmonella enteritidis, Klebsiellas including ESBL producing strains of Klebsiella pneumoniae, Acinetobacteria including without limitation Acinetobacter baumannii, Streptococcus including without limitation Streptococcus pneumoniae, Streptococcus pyogenes and Streptococcus agalactiae, Enterococcus including without limitation Enterococcus faecalis, Escherichia including without limitation Escherichia coli, particularly ESBL producing strains of Escherichia coli, blue pus bacillus (Pseudomonas aeruginosa) including without limitation MBL producing strains of Pseudomonas aeruginosa, Staphylococcus including without limitation Staphylococcus aureus, such as methicillin-resistant Staphylococcus aureus (MRSA), as well as against Haemophilus influenzae and Enterobacter including without limitation Enterobacter cloacae, in particular (without limitation) against Enterobacter cloacae strains producing expanded spectrum β-lactamases.

The data obtained during the study support the inventor assumption made within the scope of the present invention that the claimed biologically active supplement comprising extracts of Usnea barbata lichen, three-lobe beggarticks herb and common barberry berries and, optionally, extracts of black elder berries and Baikal skullcap roots, and fermented black garlic powder has the over-cumulative (synergistic) antibacterial, including bactericidal, action against the wide range of Gram-positive and Gram-negative microorganisms including antibiotic-resistant strains of Escherichia coli, Pseudomonas aeruginosa, Klebsiella pneumoniae, Enterobacter cloacae and Staphylococcus aureus and fungicidal action against Candida albicans, thereby the claimed biologically active supplement can be used not only for prevention and adjuvant therapy of the wide range of bacterial infections but also for prevention and adjuvant therapy of candida infections. These properties of the claimed biologically active supplement couldn't be predicted from the prior art.

Hypothetically, the technical results supported within the scope of the claimed invention by the above examples could be accounted by mutual potentiation of the active substances in the extracts of common barberry berries, three-lobe beggarticks herb and Usnea barbata, and optionally the Baikal skullcap root extract, elderberry berry extract, and fermented black garlic powder, which, when used as monoextracts, do not have a significant fungicidal effect. Provided technical results are unexpected, therefore the claimed invention is not obvious in view of the prior art.

1. A biologically active supplement for enhancement of the general non-specific resistance of the body and for prevention or adjuvant therapy of bacterial, fungal and viral infections, characterized in that said biologically active supplement is formulated in an oral dosage form comprising nutraceutically effective amounts of common barberry berry extract, three-lobe beggarticks herb extract, Usnea barbata lichen extract, black elder berry extract, and Baical skullcap root extract in combination with nutraceutically acceptable excipients.

2. The biologically active supplement according to claim 1, characterized in that said biologically active supplement is formulated in a tablet comprised of a film coated core, wherein said core comprises from 0.001 to 900.0 mg of common barberry berry extract, from 0.001 to 900.0 mg of three-lobe beggarticks herb extract, from 0.001 to 900.0 mg of Usnea barbata lichen extract, from 0.001 to 900.0 mg of black elder berry extract, and from 0.001 to 900.0 mg of Baical skullcap root extract in combination with nutraceutically acceptable excipients.

3. The biologically active supplement according to claim 2, characterized in that nutraceutically acceptable auxiliary excipients are selected from microcrystalline cellulose, calcium stearate or magnesium stearate, talc and amorphous (colloidal) silicon dioxide.

4. The biologically active supplement according to claim 2, characterized in that the film coat is made based on a mixture of hydroxypropyl methylcellulose, titanium dioxide, polyethylene glycol and talc.

5. The biologically active supplement according to claim 1, characterized in that said biologically active supplement is formulated in a capsule comprising from 0.001 to 900.0 mg of common barberry berry extract, from 0.001 to 900.0 mg of three-lobe beggarticks herb extract, from 0.001 to 900.0 mg of Usnea barbata lichen extract, from 0.001 to 900.0 mg of black elder berry extract, and from 0.001 to 900.0 mg of Baical skullcap root extract in combination with nutraceutically acceptable excipients.

6. The biologically active supplement according to claim 4, characterized in that nutraceutically acceptable excipients are selected from microcrystalline cellulose, calcium stearate or magnesium stearate, talc and amorphous (colloidal) silicon dioxide.

7. The biologically active supplement according to claim 1, further comprising fermented black garlic powder.

8. The biologically active supplement according to claim 1, characterized in that the bacterial infection is an infection caused by Escherichia coli including Escherichia coli strains producing expanded spectrum β-lactamases, or Staphylococcus aureus including methicillin-resistant strains of Staphylococcus aureus, or Klebsiella pneumoniae including Klebsiella pneumoniae strains producing expanded spectrum β-lactamases, or Salmonella enteritidis, or Pseudomonas aeruginosa including Pseudomonas aeruginosa strains producing metal-β-lactamases, or Enterococcus faecalis, or Acinetobacter baumannii, or Streptococcus pneumoniae, or Enterobacter cloacae including Enterobacter cloacae strains producing expanded spectrum β-lactamases, or Streptococcus pyogenes, or Haemophylus influenzae, or Streptococcus agalactiae.

9. The biologically active supplement according to claim 1, characterized in that the viral infection is influenza A, influenza B or rhinovirus infection.

10. The biologically active supplement according to claim 1, characterized in that the fungal infection is an infection caused by Candida albicans.

11. The biologically active supplement according to claim 2, further comprising fermented black garlic powder.

12. The biologically active supplement according to claim 3, further comprising fermented black garlic powder.

13. The biologically active supplement according to claim 4, further comprising fermented black garlic powder.

14. The biologically active supplement according to claim 5, further comprising fermented black garlic powder.

15. The biologically active supplement according to claim 2, characterized in that the bacterial infection is an infection caused by Escherichia coli including Escherichia coli strains producing expanded spectrum β-lactamases, or Staphylococcus aureus including methicillin-resistant strains of Staphylococcus aureus, or Klebsiella pneumoniae including Klebsiella pneumoniae strains producing expanded spectrum β-lactamases, or Salmonella enteritidis, or Pseudomonas aeruginosa including Pseudomonas aeruginosa strains producing metal-β-lactamases, or Enterococcus faecalis, or Acinetobacter baumannii, or Streptococcus pneumoniae, or Enterobacter cloacae including Enterobacter cloacae strains producing expanded spectrum β-lactamases, or Streptococcus pyogenes, or Haemophylus influenzae, or Streptococcus agalactiae.

16. The biologically active supplement according to claim 3, characterized in that the bacterial infection is an infection caused by Escherichia coli including Escherichia coli strains producing expanded spectrum β-lactamases, or Staphylococcus aureus including methicillin-resistant strains of Staphylococcus aureus, or Klebsiella pneumoniae including Klebsiella pneumoniae strains producing expanded spectrum β-lactamases, or Salmonella enteritidis, or Pseudomonas aeruginosa including Pseudomonas aeruginosa strains producing metal-β-lactamases, or Enterococcus faecalis, or Acinetobacter baumannii, or Streptococcus pneumoniae, or Enterobacter cloacae including Enterobacter cloacae strains producing expanded spectrum β-lactamases, or Streptococcus pyogenes, or Haemophylus influenzae, or Streptococcus agalactiae.

17. The biologically active supplement according to claim 2, characterized in that the viral infection is influenza A, influenza B or rhinovirus infection.

18. The biologically active supplement according to claim 3, characterized in that the viral infection is influenza A, influenza B or rhinovirus infection.

19. The biologically active supplement according to claim 2, characterized in that the fungal infection is an infection caused by Candida albicans.

20. The biologically active supplement according to claim 3, characterized in that the fungal infection is an infection caused by Candida albicans.
